(19) United States
(12) Patent Application Publication
(10) Pub. No.: US 2007/0141092 A1
(43) Pub. Date: Jun. 21, 2007

(54) BIGUANIDE COMPOSITION AND METHOD OF TREATMENT AND PREVENTION OF VIRAL INFECTIONS

(76) Inventors: Erning Xia, Penfield, NY (US); Hongna Wang, Fairport, NY (US); Lynne Brunner, Webster, NY (US); Praveen Tyle, Pittsford, NY (US)

Correspondence Address: Bausch & Lomb Incorporated, One Bausch & Lomb Place, Rochester, NY, 14604-2701, US

(21) Appl. No.: 11/610,912

(22) Filed: Dec. 14, 2006

Related U.S. Application Data
(60) Provisional application No. 60/752,455, filed on Dec. 21, 2005.
(60) Provisional application No. 60/760,510, filed on Jan. 20, 2006.
(60) Provisional application No. 60/760,880, filed on Jan. 20, 2006.
(60) Provisional application No. 60/782,478, filed on Mar. 15, 2006.
(60) Provisional application No. 60/830,319, filed on Jul. 12, 2006.
(60) Provisional application No. 60/830,326, filed on Jul. 12, 2006.

(51) Int. Cl. A61K 31/155; A61K 9/00

(52) U.S. Cl. 424/400; 514/635

(57) ABSTRACT

An ophthalmically acceptable composition comprising to the ocular region of a patient, the ophthalmically acceptable composition comprising water, a biguanide containing antimicrobial in an amount effective to treat viral infection. The invention further comprises administering the ophthalmically acceptable composition to the eye of a patient in need of treatment.

### CROSS REFERENCE TO RELATED APPLICATIONS

This application claims priority to U.S. Provisional Patent Application Nos. 60/752,455 filed Dec. 21, 2005; 60/760,510 filed Jan. 20, 2006; 60/760,880 filed Jan. 20, 2006; 60/782,478 filed Mar. 15, 2006; 60/830,319 filed Jul. 12, 2006 and 60/830,326 filed Jul. 12, 2006; the contents of each being incorporated by reference herein.

### FIELD OF THE INVENTION

This invention relates to the treatment of viral infections with topical formulations.

### DISCUSSION OF THE RELATED ART

Viral infections are often highly infectious, rapidly mutating, and often debilitating. One type of viral disease is Herpes simplex—commonly referred to as cold sores or fever blisters. Herpes is a viral infection that causes lesions on the tissue of the infected such as blisters and sores. One feature of a virus is its potential for spread and reoccurrence. It is believed that when treated, the Herpes virus is never completely removed from the body, but resides and potentially spreads along the nervous system. For example a herpes virus outbreak that originally resides in the mouth can potentially spread along the nervous system to the eye or other parts of the face.

Herpes infections are very common. It is estimated that 90% of the population have been exposed to herpes—the most common outbreaks of herpes around the mucosal membranes of the mouth or genital region. Ocular herpes is relatively rare, but difficult to treat. When the eye is afflicted by herpes simplex, it usually affects only one eye and most often occurs on the cornea of the eye. This type of corneal infection is called Herpes Keratitis. The infection may be superficial, involving only the top layer of the cornea—referred to as the epithelium. Generally the lesions on the eye will heal without scarring. However, when the infection involves deeper layers of the cornea, it may lead to scars of the cornea, loss of vision, and sometimes even blindness. Less commonly, herpes simplex virus may also infect the inside of the eye (Herpes Uveitis) or the retina (Herpes Retinitis).

Current treatment for Herpes and other ocular viral disease may include administering systemic antiviral agents. One type of systemic antiviral agents have viral thymidine kinase activity. Viral thymidine kinase converts these drugs to a monophosphate form which disrupts replication of the virus. Examples of such include Valaciclovir (GlaxoSmithKline, Philadelphia, Pa.) disclosed in U.S. Pat. No. 4,957,924; Famciclovir (Novartis, East Hanover, N.J.) covered in U.S. Pat. No. 5,246,937; Tromantadine and Penciclovir (GlaxoSmithKline, Philadelphia, Pa.) disclosed in U.S. Pat. No. 5,075,445.

Another class of systemic treatment prevents the virus from attaching to cell membranes and thus, barring entry of the virus DNA to the host cell. This treatment method is effective for containing an outbreak of Herpes. Doccsanol sold under the trademark Abreva (GlaxoSmithKline, Philadelphia) is sold in a 10% topical cream form. U.S. Pat. No. 4,874,794 relates to doccsanol products.

Patients with topical virus infections may benefit from a topically administered antiviral ointment. Treatment of topical virus infections with a topical ointment compared to a systemic antiviral medicament will limit any toxicity of the medicine and other side effects because therapeutic levels of the antiviral agent is not required throughout the entire body. Ophthalmic ointments for treatment of ocular disease include but are not limited to Acyclovir ophthalmic ointment (GlaxoSmithKline, Philadelphia, Pa.) or Viroptic® 1.0% sterile ophthalmic solution of trifluridine (King Pharmaceuticals, Bristol, Tenn.).

For more developed infection, some ophthalmologists may also treat these patients by wiping away infected cells from the cornea with a dry, cotton-tipped applicator. Treatment may vary for deeper, more severe corneal infection and for herpetic inflammation within the eye. The antiviral eye drops presently available are less effective in treating these severe infections than early stage infections. Steroids, in the form of drops, may help decrease inflammation and corneal scarring. Despite the available treatments, some patients do not respond well or rapidly to treatment. These patients may have prolonged inflammation and ultimately permanent corneal scarring and may need corneal transplantation to restore their vision. Thus, better therapies for viral infection, including topical viral infection and particularly ocular viral infection, are required.

Biguanide antimicrobial agents have been used to preserve ophthalmic solutions and demonstrate relatively low toxicity in ocular tissues. Biguanide antimicrobial agents include polyhexamethylene biguanide, chlorhexidine and Alexidine.

To effectively preserve an ophthalmic composition, sufficient preservative is necessary to prevent growth of S. aureus, P. aeruginosa and E. coli bacteria and C. albicans and A. niger fungi over the shelf life of the product. Typically, a clinically effective formulation will contain an amount of a preservative required to accomplish the preservative effect without unnecessary excess. Between 0.5 ppm and 3.0 ppm of a biguanide has been used to preserve most ophthalmic solutions.

Biguanide antimicrobial agents have been used as disinfectant agents for contact lenses. To be considered a disinfectant, a solution needs sufficient antimicrobial agent to kill S. aureus, P. aeruginosa and S. marcescens bacteria and C. albicans and F. solani fungi over the shelf life of the product. Furthermore, the solution must show efficacy in disinfecting contact lenses using the disinfecting regimen that is recommended on the product. This regimen is arrived at through data which supports the disinfecting properties described above.

Disinfecting solutions containing antimicrobial agents include ReNu® Multiplus sold by Bausch & Lomb, Rochester, N.Y. ReNu® Multiplus is a multipurpose cleaning, conditioning and disinfecting solution for contact lenses that contains 1 ppm of polyhexamethylene biguanide. ReNu® with MoistureLoc is a multipurpose cleaning, conditioning and disinfecting solution for contact lenses that contains 4.5 ppm of Alexidine.

Disinfecting solutions such as the two mentioned above are ophthalmically safe solutions. They are safe to administer to the eye of a patient. Contact lenses that have been rinsed with these solutions are placed in the eye. However, these solutions are not approved for use as a medicament in the eye. There is no evidence to suggest that the level of antimicrobial agent in a multipurpose contact lens solution would be effective to treat ocular infection.

Several studies have been conducted on the effectiveness of polyhexamethylene biguanide and/or chlorhexidine for treatment of Acanthamoebal keratitis.

In Schuster, et al., “Opportunistic Amoebae: Challenges In Prophylaxis And Treatment,” Drug Resistance Updates: Reviews And Commentaries In Antimicrobial And Anticancer Chemotherapy, vol. 7(1) pp. 41-51 (February 2004), Acanthamoeba keratitis, a non-opportunistic infection of the cornea, was found to respond to treatment with chlorhexidine gluconate and polyhexamethylene biguanide, in combination with propamidine isothionate (Brolene), hexamidine (Desomodine), or neomycin.

In Rama et al., “Bilateral Acanthamoeba keratitis with late recurrence of the infection in a corneal graft: a case report,” European Journal of Ophthalmology, vol. 13 (3), pp. 311-4 (April 2003), recurrences of Acanthamoeba keratitis in both eyes were successfully treated with a combination of hexamidine and neomycin, and with polyhexamethylene biguanide, respectively.

Anita et al., “Role of 0.02% polyhexamethylene biguanide and 1% povidone iodine in experimental Aspergillus keratitis,” Cornea, Vol. 22 (2), pp. 138-41, (March 2003) showed that polyhexamethylene biguanide (0.02%) is a moderately effective drug for experimental Aspergillus keratitis.

Sharma et al., “Patient characteristics, diagnosis and treatment of non-contact lens related Acanthamoeba keratitis,” British Journal of Ophthalmology, Vol. 84/10, pp. 1103-1108 (2000) illustrates the combination of polyhexamethylene biguanide and chlorhexidine.

Alexidine has been screened against Acanthamoeba keratitis in several studies. See Eye, vol. 17, pp. 893-905 (2003). J. Pharm. Pharmacol. (47, No. 12B, 1107, 1995) 1 Tab. 6 Ref. British Journal of Ophthalmology, (1996) Vol. 80, No. 9, pp. 849. Transactions of the Royal Society of Tropical Medicine and Hygiene (1995) 89, 245-247.

U.S. Pat. No. 5,942,218 teaches the use of an anti-infective material based upon polyhexamethylene biguanide as a component in an antiviral composition that can be used for wound treatment.

Consequently, there is a need for a topical antimicrobial composition that is effective treatment for viral infections. Additionally, there is a need for a topical ophthalmic antimicrobial composition that is effective for treatment of viral infections in the ocular region of the patient. The present invention addresses these and other needs.

### SUMMARY OF INVENTION

The present invention, according to one embodiment, is a method of treating a viral infection comprising administering a topical composition to the skin or mucous membranes of a patient. The topical composition comprises a topically acceptable carrier and a biguanide containing antimicrobial agent.

The present invention, according to one embodiment, is a method of treating a viral infection comprising administering an ophthalmically acceptable composition to the ocular region of a patient, the ophthalmically acceptable composition comprising an ophthalmically acceptable carrier and a biguanide containing antimicrobial agent. The administration of the biguanide antimicrobial agent to the eye results in a reduction of the viral load in the eye. Typically, the administration of the biguanide antimicrobial agent results in a reduction of the viral load to the extent that the symptoms of the viral infection are reduced or, preferably eliminated. The topically or ophthalmically acceptable carrier is water containing carrier. In another embodiment, the topically or ophthalmically acceptable carrier is an oil, grease, wax or petrolatum based carrier.

The present invention, according to one embodiment, is administered to the ocular region of a patient. Typically, the ophthalmically acceptable composition can safely be administered to the eye of a patient. By safe, it is meant that the medicament is approved for use in the eye or is capable of being approved for use in the eye by the Food and Drug Administration. The medicament does not contain any ingredients that are toxic or harmful or cause an unacceptable degree of irritation to the eye of a patient according to FDA guidelines.

In another embodiment, the method includes treating a patient that is infected with a viral infection. In another embodiment, the method includes treating a patient that is infected with the Herpes virus. Typically, the patient is infected with Herpes Simplex-1. In another embodiment, the patient is infected with Herpes Simplex-2. In still another embodiment, the patient is infected with an adenovirus. In still another embodiment the adenovirus is Adenovirus Type-4 or Adenovirus Type-8. In one other embodiment, the virus is cytomegalovirus.

In another embodiment, there is a composition for treating infectious disease comprising water, and a biguanide containing antimicrobial agent in an amount effective to treat a viral infection.

### DETAILED DESCRIPTION OF THE PREFERRED EMBODIMENT

Alexidine is a biguanide antimicrobial agent that is defined by the formula 1,1′-hexamethylene-bis[5-(2-ethylhexyl)biguanide]. By biguanide antimicrobial agent it is meant an antimicrobial agent that has biguanide substituents and has antimicrobial properties in an ophthalmically safe amount. Suitable biguanide antimicrobial agents include but are not limited to 1,1′-hexamethylene-bis[5-(p-chlorophenyl)biguanide](Chlorhexidine) or water soluble salts thereof, 1,1′-hexamethylene-bis[5-(2-ethylhexyl)biguanide](Alexidine) or water-soluble salts thereof, and poly(hexamethylene biguanide) (PHMB).

In one embodiment, the amount of antimicrobial agent in the ophthalmic composition is a minimum of about 1 ppm and a maximum of about 10 wt. %. Typically, the amount of antimicrobial agent in the ophthalmic composition is a minimum of about 5 ppm, about 10 ppm, about 20 ppm, about 50 ppm, about 100 ppm or about 200 ppm. Typically, the amount of antimicrobial agent in the ophthlamic composition is a maximum of about 1 wt. %, 1000 ppm, about 500 ppm, about 300 ppm, about 100 ppm. In one embodiment, the amount of Alexidine is about 30 ppm. In another embodiment, the amount of Alexidine is about 300 ppm.

Due to the tendency of Alexidine or other biguanide antimicrobial agents to hydrolyze in an aqueous solution, it is desirable to include a stabilizer for formulations in which Alexidine is likely to hydrolyze. A stabilizer is a compound that prevents the chemical degradation of an active agent when the compound is in the presence of the stabilizer. Examples of stabilizers that are effective in an aqueous solution include but are not limited to hydroxyl alkyl phosphonate, Tetronics® 908, tyloxapol, cyclodextrin and derivatives of cyclodextrin, hyaluronic acid, sodium edetate, citric acid as well as ophthalmically acceptable antioxidants, complexing agents and chelating agents and salts thereof. In one embodiment, preferred stabilizers are hydroxyalkyl phosphonate, ethylenediamine-tetraacetic acid, Tetronics® 908, tyloxapol, cyclodextrin and derivatives of cyclodextrin, hyaluronic acid or EDTA.

In one embodiment, the stabilizer is present in an amount effective to stabilize the compound. An amount effective to stabilize a compound means that the stabilizer is present in an amount that prevents deterioration of at least 90% of the compound in a period of 24 months. In another embodiment, the preferred stabilizer is present in a minimum amount of about 0.001 wt. %, about 0.005 wt. %, about 0.01 wt. % and/or a maximum amount of about 5 wt. %, about 1 wt. %, about 0.5 wt. %, about 0.3 wt. %, about 0.1 wt. %, about 0.08 wt. %, about 0.05 wt. %, about 0.03 wt. %, about 0.01 wt. % based upon the total volume of the composition. In another embodiment, the stabilizer is a cyclodextrin or cyclodextrin derivative and is present in an amount that is a minimum of about 0.001 wt. %, about 0.005 wt. %, about 0.01 wt. % and/or a maximum of about 50 wt. %, about 40 wt. %, about 20 wt. % or about 10 wt. % cyclodextrin or cyclodextrin derivative based upon the total amount of the composition.

In another embodiment the effective shelf life of the antimicrobial agent is extended by a minimum of about 10 percent of the shelf life without the stabilizer. In another embodiment, the antimicrobial agent is extended by a minimum of about 20 percent, about 40 percent, about 80 percent, about 100 percent or about 200 percent.

Delivery Vehicle

In another embodiment, the composition of the present invention contains a delivery vehicle that increases the mean residence time of the active agent in the eye and/or enhances penetration in the eye. U.S. Pat. Nos. 6,884,788 or 6,261,547 or 5,800,807 or 5,618,800 or 5,496,811 disclose various ophthalmic delivery vehicles the teachings in these patents are incorporated by reference in their entirety.

Various anatomical barriers relating to the eye may underlie the poor intraocular penetrance of whole antibodies. In this regard, the cornea is the principal barrier to entry of foreign substances. It has two distinct penetration barriers, the corneal epithelium and the corneal stroma. Thus, it is desirable to use a penetration enhancer to improve the penetration of the active ingredients of the present invention.

The penetration enhancer generally acts to make the cell membranes less rigid and therefore more amenable to allowing passage of drug molecules between cells. The penetration enhancers preferably exert their penetration enhancing effect immediately upon application to the eye and maintain this effect for a period of approximately five to ten minutes. The penetration enhancers and any metabolites thereof must also be non-toxic to ophthalmic tissues. One or more penetration enhancers will generally be utilized in a minimum amount of about 0.01 weight percent and/or a maximum of about 10 wt. %.

The preferred penetration enhancers are saccharide surfactants, such as dodecylmaltoside (“DDM”), and monoacyl phosphoglycerides, such as lysophosphatidylcholine. The saccharide surfactants and monoacyl phosphoglycerides, which may be utilized, as penetration enhancers in the present invention are known compounds. The use of such compounds to enhance the penetration of ophthalmic drugs is described in U.S. Pat. No. 5,221,696 the entire contents of which are incorporated by reference into the present specification.

The viscosifiers are optionally used in the present invention to increase the mean residence time of the active ingredient in the eye. With the aid of a viscosifier, liquid drops can be used having a viscosity that is a minimum of about 2 cps and a maximum of about 100 cps. Viscosifiers can be used to formulate liquid gels that have a viscosity that is a minimum of about 100 cps and a maximum of about 1000 cps. Ophthalmic gels will generally have a viscosity in excess of about 1,000 cps. Regardless, the viscosifier is utilized to ensure an adequate mean residence time in the eye. Any synthetic or natural polymer, which is capable of forming a viscous or a solid insert, may be utilized. In addition to having the physical properties required to form a viscous gel or solid insert, the polymers must also be compatible with tissues of the eye. The polymers must also be chemically and physically compatible with the above-described active agent and other components of the compositions.

Polymers, which satisfy the foregoing criteria, are referred to herein as “ophthalmically acceptable viscous polymers.” Examples of suitable polymers include: natural polysaccharides and gums, such as alginate, carrageenan, guar, karaya, locust bean, tragacanth agarose and xanthan; modified naturally occurring polymers such as carboxymethylcellulose, hydroxyethylcellulose, hydroxypropylcellulose, hydroxypropylmethylcellulose, methylcellulose, hydroxypropylmethylguar and carboxymethyguar, synthetic polymers, such as carboxy vinyl polymers, polyvinyl alcohol and polyvinyl pyrrolidone.

In addition, proteins and synthetic polypeptides that form viscous gels and are ophthalmically acceptable can be used to provide better bioavailability. Typically, proteins that can be used include: gelatin, collagen, albumin, whey protein and casein.

Polymers which have high molecular weights and, most importantly, physical properties that mimic the physical properties of the mucous secretions found in the eye are referred to herein as being “mucomimetic.” A preferred class of mucomimetic polymers are carboxy vinyl polymers having molecular weights in the range of from about 50,000 to about 6,000,000. The polymers have carboxylic acid functional groups and preferably contain between 2 and 7 carbon atoms per functional group. The gels that form during preparation of the ophthalmic polymer dispersion have a viscosity between about 1,000 to about 300,000 centipoise (cps). Suitable carboxy vinyl polymers include those called carbomers, e.g., Carbopol® P (B.F. Goodrich Co., Cleveland, Ohio). Specifically preferred are carbomer 934, 940, 970, 974 and 980. Such polymers will typically be employed in an amount between about 0.05 and about 8.0 wt %, depending on the desired viscosity of the composition.

Aqueous compositions of the invention have an ophthalmically compatible pH, which generally will range between about 6 to about 8, and more preferably between 6.5 to 7.8, and most preferably about 7 to 7.5. One or more conventional buffers may be employed to obtain the desired pH value. Suitable buffers include for example but are not limited to borate buffers based on boric acid and/or sodium borate, phosphate buffers based on Na₂HPO₄, NaH₂PO₄ and/or KH₂PO₄, citrate buffers based on sodium or potassium citrate and/or citric acid, succinate buffers, sodium bicarbonate, aminoalcohol buffers, Good buffers and combinations thereof. Generally, buffers will be used in amounts ranging from about 0.05 to about 2.5 weight percent, and preferably, from about 0.1 to about 1.5 weight percent.

Compositions of the present invention likewise include one or more tonicity agents to approximate the osmotic pressure of normal lachrymal fluids, which is equivalent to a 0.9 percent solution of sodium chloride or 2.5 percent glycerin solution. Examples of suitable tonicity agents include but are not limited to sodium and potassium chloride, dextrose, mannose, glycerin, calcium and magnesium chloride. These agents are typically used individually in amounts that are a minimum of about 0.01 wt. % or about 0.2 wt. % and/or a maximum of about 2.5 wt. % or 1.5 wt. %. Preferably, the tonicity agent is employed in an amount to provide a final osmotic value that is a minimum of about 200 mOsm/kg, about 220 mOsm/kg and/or a maximum of about 450 mOsm/kg, about 350 mOsm/kg or about 320 mOsm/kg.

Aqueous compositions may likewise include a humectant to provide moisture to the eye. A first class of humectants is polymer humectants. Examples of suitable humectants include for example but are not limited to poly(vinyl alcohol) (PVA), poly(N-vinylpyrrolidone) (PVP), cellulose derivatives and poly(ethylene glycol). As disclosed in U.S. Pat. No. 6,274,133, cationic cellulosic polymers include for example but are not limited to water soluble polymers commercially available under the CTFA (Cosmetic, Toiletry, and Fragrance Association) designation Polyquaternium-10, including the cationic cellulosic polymers available under the trade name UCARE® Polymers from Amerchol Corp., Edison, N.J., such as for example but not limited to Polymer JR™. Generally, these cationic cellulose polymers contain quaternized N,N-dimethylamino groups along the cellulosic polymer chain.

Another suitable class of humectants is non-polymeric humectants. Examples may include glycerin, propylene glycol, and other non-polymeric diols and glycols. The specific quantities of humectants used in the invention will vary depending upon the application. However, the humectants will typically be included in an amount from about 0.01 to about 5 weight percent, preferably from about 0.1 to about 2 weight percent.

It will be understood that some constituents possess more than one functional attribute. For example, cellulose derivatives are suitable polymeric humectants, but are also referred to as “viscosity increasing agents” to increase viscosity of the composition if desired. Glycerin is a suitable non-polymeric humectant but is also may contribute to adjusting tonicity.

Compositions of the present invention may optionally include one or more sequestering agents. Suitable sequestering agents include for example but are not limited to ethylenediaminetetraacetic acid (EDTA) and its salts. Sequestering agents are preferably present in a minimum of about 0.01 wt. % and/or a maximum of about 0.2 wt. %.

It will be understood that the present invention is typically applied by administering a composition to the eye of a patient in the form of eye drops, liquid gels or viscous gels. In one embodiment, one to four drops are applied to each eye. Preferably two drops are applied to each eye. In one embodiment, the drops are placed directly on the eye. In another embodiment, the drops are placed in the conjuntival sac beneath the eye.

Typically, the drops are administered a minimum of once daily, two times daily or three times daily.

### EXAMPLES

### Example 1

### HSV-1 Viral Suspension Assay

The Viral Suspension Assay was used to evaluate the antiviral properties of Alexidine against Herpes simplex virus type 1 when exposed in suspension for 1, 2, 5, and 10 minutes. The presence of virus (infectivity) was determined by monitoring the virus specific cytopathic effect (CPE) on an appropriate indicator cell line, rabbit kidney. Results are reported as Percent (%) Reduction in virus titer as compared to the corresponding virus control titer (Table 1). The titer of the virus controls were 7.5 log₁₀ following the one minute exposure time; 7.0 log₁₀ following the two minute exposure time; and 7.75 log₁₀ following both the five and ten minute exposure times. The results are listed in Table 1 and show that Alexidine at both 30 ppm and 99 ppm are effective agents against herpes simplex type-1 virus (HSV-1).

TABLE 1
Viral Suspension Assay Percent Reduction of Herpes simplex virus
type 1 after 1, 2, 5 and 10 Minute Exposure to Alexidine

| Alexidine | | | | |
|---|---|---|---|---|
| Test Concentration | 1 minute | 2 minutes | 5 minutes | 10 minutes |
| 30 ppm | 99.99% | 99.99% | 99.9994% | ≧99.99994% |
| 99 ppm | 99.999% | 99.994% | 99.9999% | ≧99.99994% |

### Example 2

### Adenovirus and Cytomegalovirus Testing

The Viral Suspension Assay was used to evaluate the antiviral properties of Alexidine against Adenovirus Type-4, Adenovirus Type-8 and Adenovirus Type-19 and Cytomegalovirus when exposed in suspension for 1, 2, 5, and 10 minutes. The presence of virus (infectivity) was determined by monitoring the virus specific cytopathic effect (CPE) on an appropriate indicator cell line, rabbit kidney. Results are reported as Percent (%) Reduction in virus titer as compared to the corresponding virus control titer (Table 1). The titer of the virus controls were 7.5 log₁₀ following the one minute exposure time; 7.0 log₁₀ following the two minute exposure time; and 7.75 log₁₀ following both the five and ten minute exposure times. The results are listed in Table 1 and show that Alexidine at both 30 ppm and 99 ppm are somewhat effective against viral strains of Adenovirus Type-4, Adenovirus Type-8, and Cytomegalovirus. However, Alexidine did not appear to be effective against the particular strain of Adenovirus Type-19 that was tested. Alexidine is a potent antimicrobial agent against Herpes Simplex-1 and has some effectiveness against certain strains of other viruses that cause ocular infection.

TABLE 2
Viral Suspension Assay Percent Reduction of Adenovirus Type-4,
Adenovirus Type-8 and Adenovirus Type-19 and Cytomegalovirus
after 1, 2, 5 and 10 Minute Exposure to Alexidine

| | Alexidine | Percent Reduction (%) | | | |
|---|---|---|---|---|---|
| | Test | 1 | 2 | 5 | 10 |
| Virus | Concentration | minute | minutes | minutes | minutes |
| Adenovirus | 30 ppm | 43.8 | — | 82.2 | 68.4 |
| type 4 | 99 ppm | 68.4 | — | 43.8 | 68.4 |
| Adenovirus | 30 ppm | 96.8 | 94.4 | 82.2 | 90.0 |
| type 8 | 99 ppm | 82.2 | 82.2 | 90.0 | 90.0 |
| Adenovirus | 30 ppm | No reduction | | | |
| type 19 | 99 ppm | | | | |
| Cytomegalovirus | 30 ppm | 43.8 | 68.4 | — | 43.8 |
| | 99 ppm | 98.2 | 99.0 | 99.8 | 99.98 |

What is claimed is:

1. A method of treating a viral infection comprising administering an ophthalmically acceptable composition to the ocular region of a patient, the ophthalmically acceptable composition comprising an ophthalmically acceptable carrier and a biguanide containing antimicrobial agent.

2. The method of claim 1, wherein the composition further comprises a penetration enhancer.

3. The method of claim 2, wherein the penetration enhancer is present in an amount that is a minimum of about 0.001 wt. % and a maximum of about 5 wt. %.

4. The method of claim 1, wherein the ophthalmically acceptable carrier is water.

5. The method of claim 1, wherein the composition further comprises a stabilizer in an amount effective to extend the shelf-life of the stabilizer a minimum of about 10% of the shelf-life of a similar composition without the stabilizer.

6. The method of claim 1, wherein the composition further comprises a viscosifier.

7. The method of claim 5, wherein the viscosifiers are selected from the group consisting of natural polysaccharides, natural gums, modified natural polymers, synthetic polymers, proteins and synthetic polypeptides that are capable of increasing viscosity and are ophthalmically acceptable.

8. The method of claim 6, wherein the viscosifiers are selected from the group consisting of mucomimetics.

9. The method of claim 6, wherein the viscosifier is a carboxyvinyl polymer.

10. The method of claim 1, wherein the biguanide antimicrobial agent is present in an amount ranging from 1 ppm to about 1.0 wt. % based upon the total amount the composition.

11. The method of claim 1, wherein the infectious disease is herpes.

12. The method of claim 1, wherein the infectious disease is HSV-1 type herpes.

13. The method of claim 1, wherein the infectious disease is HSV-2 type herpes.

14. The method of claim 1, further comprising a stabilizer.

15. A composition for treating infectious disease comprising water, and a biguanide containing antimicrobial agent in an amount effective to treat a viral infection.

16. A method of topically treating a viral infection comprising:
administering to a patient diagnosed with a topical viral infection, a composition comprising a topically acceptable carrier and an antiviral effective amount of Alexidine.

17. The method of claim 16, wherein the composition further comprises a penetration enhancer.

18. The method of claim 17, wherein the penetration enhancer is present in an amount that is a minimum of about 0.001 wt. % and a maximum of about 5 wt. %.

19. The method of claim 16, wherein the topically acceptable carrier is an oil, wax, grease or petrolatum containing carrier.

20. The method of claim 16, further comprising a stabilizer is in an amount effective to extend the shelf life a minimum of about 10% of the shelf-life without the stabilizer.

21. The method of claim 16, wherein the composition further comprises a viscosifier.

22. The method of claim 22, wherein the viscosifier is selected from the group consisting of natural polysaccharides, natural gums, modified natural polymers, synthetic polymers, proteins and synthetic polypeptides that are capable of increasing viscosity and are ophthalmically acceptable.

23. The method of claim 22, wherein the viscosifier is selected from the group consisting of mucomimetics.

24. The method of claim 22, wherein the viscosifier is a carboxyvinyl polymer.

25. The method of claim 16, wherein the Alexidine is present in an amount ranging from 1 ppm to about 1.0 wt. % based upon the total amount the composition.

26. The method of claim 16, wherein the infectious disease is herpes.

27. The method of claim 16, wherein the infectious disease is HSV-1 type herpes.

28. The method of claim 16, wherein the infectious disease is HSV-2 type herpes.
